(19) United States
(12) Patent Application Publication
(10) Pub. No.: US 2005/0061332 A1
(43) Pub. Date: Mar. 24, 2005

(54) Method and apparatus for scanning feet for the purpose of manufacturing orthotics and other footwear

(76) Inventors: Kent S. Greenawalt, Roanoke, VA (US); Dwayne H. Bennett, Roanoke, VA (US)

Correspondence Address: STURM & FIX LLP, 206 SIXTH AVENUE, SUITE 1213, DES MOINES, IA, 50309-4076, US

(21) Appl. No.: 10/668,835

(22) Filed: Sep. 23, 2003

(51) Int. Cl. A61F 5/37

(52) U.S. Cl. 128/882

(57) ABSTRACT

Measurements for corrective footwear, orthopedics, and orthotics have traditionally been obtained through three-dimensional casts of a foot. Digital technology opens an avenue of utilizing a digital image in place of the cast. Using a single or dual flatbed scanner and a computer, all the data required to construct a corrective device may be obtained from a scan or scans of the foot. Observed features and color variations, as well as physical measurements are used to overcome imperfections of a patient's foot.

### BACKGROUND OF THE INVENTION

1. Field of the Invention

This invention relates generally to a method and apparatus for obtaining foot images for use in making orthopedic appliances. More particularly, the present invention is connected with the use of a flat-bed scanner for obtaining foot measurements for the making of arch supports, orthotics, custom shoes, and custom sandals.

2. Background Art

In the making of orthopedic appliances such as custom orthotics, custom shoes and custom sandals, all referred to herein as “corrective orthotics,” to aid in the correction or prevention of malformations of the feet, it is necessary to first obtain an image of the foot. Measurements are then taken of the image for the purpose of duplicating the contours of the foot. For accuracy, the foot image must be taken while the foot is positioned in a normal weight bearing condition. The conventional practice, which has remained unchanged for years, is to make a depression with the foot. Plaster of Paris is then poured into the negative depression, producing a duplicate mold or cast of the bottom of the foot. From the molded form, which provides an exact duplication of the contours of the foot, an orthopedic device such as an arch support or an entire shoe is made. One material used for making the negative image is a foam that is in a box. The patient steps into the foam to make an impression of the foot.

The present methods of producing an image of a foot require a significant amount of material and equipment in terms of volume and mass. Additionally, the casts produced are voluminous, requiring significant storage space. Transfer of the casts to another location requires significant effort and expense as well.

There is, therefore, a need for a method and device for acquiring measurements and data necessary for producing orthotics and corrective shoes and sandals using electronic data that can easily be stored and transferred over common communication pathways.

### SUMMARY OF THE INVENTION

It is an object of this invention to provide a novel method and apparatus for obtaining foot measurements for the making of corrective footwear such as arch supports, orthotics, and corrective shoes and sandals. It is a further purpose of the present invention to produce data needed for the necessary measurements in electronic format for ease of storage and transmittal.

Modern electronic technology has made it possible to utilize a flatbed scanner to record a color image of a patient's foot, from which the necessary measurements and other observations may be obtained. The measurements may be used to produce corrective orthotics, shoes, or sandals. The corrective support in any of these products does not mirror the contours of the foot, but rather restores normal foot balance.

Another object of this invention is to improve on known methods of making arch supports, by providing a method and apparatus by which accurate measurements, visual observations, and color variations of an electronic image (or a hardcopy of an electronic image) serve as a basis for a corrective arch support. This approach eliminates a step that can be rather messy and is a sometimes harmful process involving plaster of Paris.

Yet another object of this invention is to provide an improved method of using foot images to make corrective arch supports, which method includes the use of scans which lend themselves to an instantaneous image.

It is another object of this invention to provide a novel method of obtaining foot measurements as described herein before which may be performed easily and with a minimum of time and equipment, and which is effective and accurate.

The novel features which are believed to be characteristic of this invention, both as to its organization and method operation together with further objectives and advantages thereto, will be better understood from the following description considered in connection with accompanying drawings in which a presently preferred embodiment of the invention is illustrated by way of example. It is to be expressly understood however, that the drawings are for the purpose of illustration and description only and not intended as a definition of the limits of the invention.

### BRIEF DESCRIPTION OF THE DRAWINGS

FIG. 1 shows a computer and a flatbed scanner.

FIG. 2 show two flatbed scanners in use scanning feet.

FIG. 3 show flow diagrams of foot scanning for single and dual scanner systems.

FIG. 4 depicts color scans of two feet.

FIG. 5 shows a line drawing of a foot.

FIG. 6 shows an orthotic.

FIG. 7 depicts a color scan of a foot and a ruler for making measurements.

### BEST MODE FOR CARRYING OUT THE INVENTION

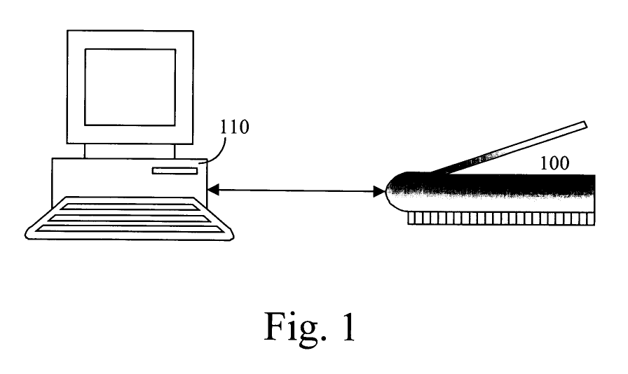

A flatbed scanner 100, depicted in FIG. 1 is used in the present invention to obtain electronic color images of a patient's feet. The scanner is connected, electronically, to a personal computer 110. Image processing software (see Appendix) within the computer 110 organizes the data to present them most conveniently to an operator.

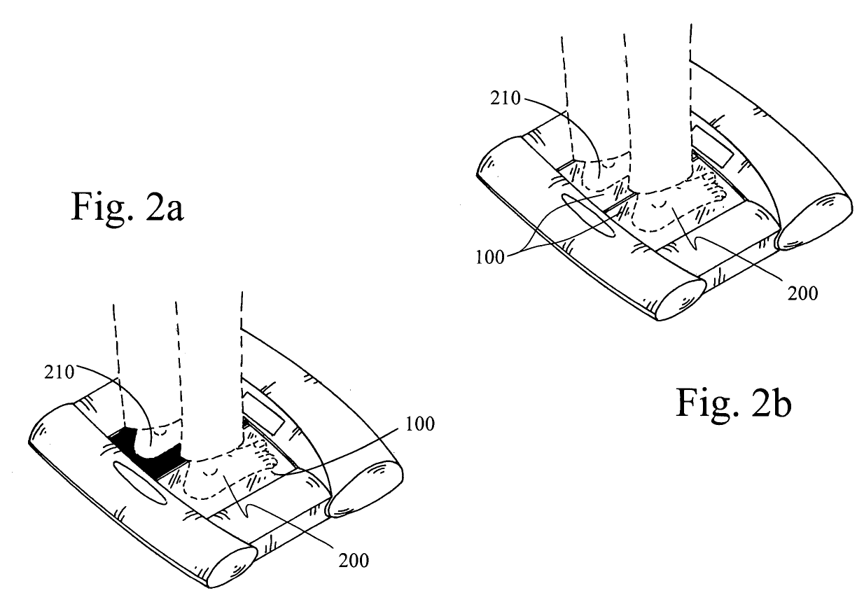

In FIG. 2, representative configurations for flatbed scanners 100 are shown with a patient standing thereon. In FIG. 2a, a single-scanner unit is shown. Only the right foot 200, in this instance, is on the scanner 100 and being scanned. The left foot 210 is on a surface that is at the same elevation as the flatbed scanner 100 so the patient may stand normally. The left foot 210 will be scanned after the patient is repositioned with the left foot 210 on the flatbed scanner 100. In FIG. 2b, both feet 200, 210 are on scanners 100. The feet 200, 210 may be scanned simultaneously or sequentially. The present invention is not limited to the physical configurations of the scanners 100 shown in FIG. 2.

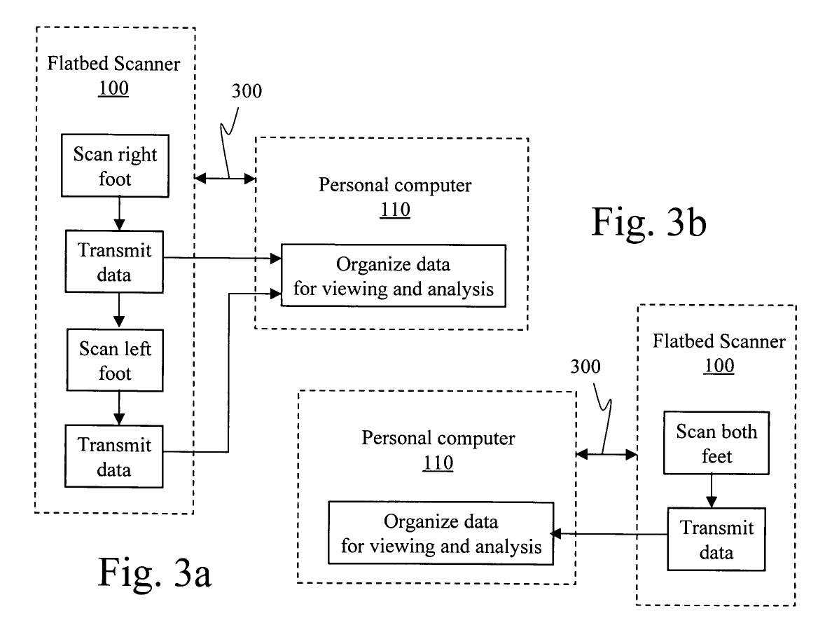

Flow diagrams for the scanning process are depicted in FIG. 3. Focusing on FIG. 3a, the flatbed scanner(s) 100 is shown on the left while a personal computer 110 is shown on the right. Scanning control communication information 300 is passed between the personal computer 110 and the scanner(s) 100. This passing of control information is the same as passed between any computer 110 and scanner 100 used for any purpose and includes commands from the computer 110 to begin or abort a scan, and signals from the scanner 100 indicating it has finished a scan and is ready to perform another.

FIG. 3a represents either a single scanner operation or a dual scanner operation. In either case, the patient's feet are scanned one at a time. In FIG. 3a, the right foot 200 is shown being scanned first, but this invention is not limited to any particular order. After each foot is scanned, the data are transmitted to the computer 110 where software included on a CD with this application (see the Appendix for a directory listing) organizes them for ease of viewing and for the purpose of retrieving the measurements required to construct corrective footwear.

In FIG. 3b, a single or dual scanner system is depicted wherein both feet are scanned simultaneously and the data transferred to the computer 110 where the data are sorted for presentation and measurement.

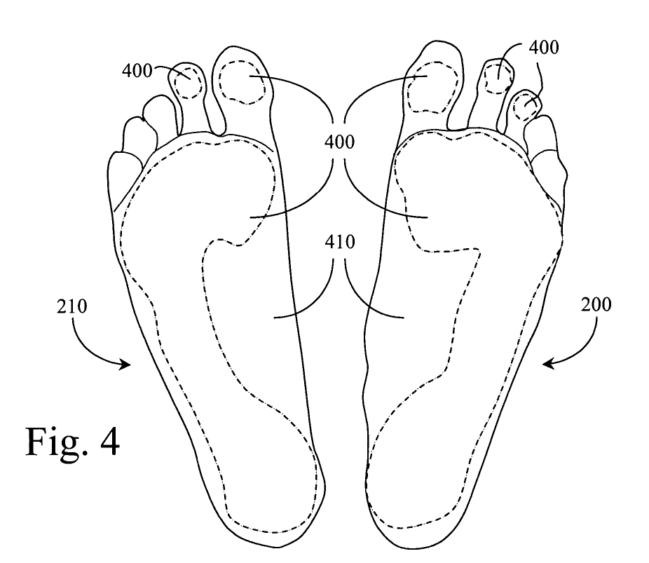

FIG. 4 represents a pair of color images of feet as taken with a scanner. These images are captured by the scanner(s) and, through software, are displayed side by side on the screen as seen in FIG. 4.

In FIG. 4, the right foot 200 is on the right side and left foot 210 is on the left. The images are actually inverted so they appear on the predetermined side without being mirrored (or backwards). Size, dimensions, color variations, texture variations, and abnormalities are clearly visible in a typical image taken by a scanner. Depth perception is achieved by variations in colors and shading. Images closest to surface show as light tones 400 while areas further away darken 410. Much more detail may be discerned in an actual scan than is depicted in FIG. 4.

The method of obtaining measurements for making a pair of arch supports is as follows. Place a foot 200, 210 on the scanner 100, the foot being positioned such that the entire foot will appear in the scanned image. The patient shall stand so that his or her weight is divided normally between both feet. If a single scanner 100 is employed (see FIG. 2a), the first foot is moved out of the window upon the completion of the first scan and the second foot is then placed on the scanner surface as the image is captured. If a dual scanner 100 is used (see FIG. 2b), it is not necessary to move the feet. These measurements and observations are then corrected for the building of corrective arch supports and like orthopedic devices, orthotics, shoes and sandals.

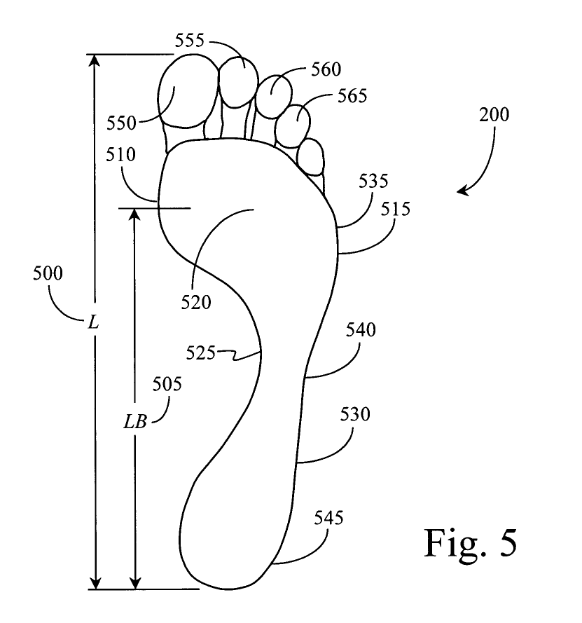

In FIG. 5, an outline primarily of the lowest surface of the right foot 200 is depicted. This image will be used to describe specific dimensions and measurements used in designing corrective footwear. While the following measurements and observations are made of the right foot 200, it is understood that the same measurements and observations will be made for the left foot 210.

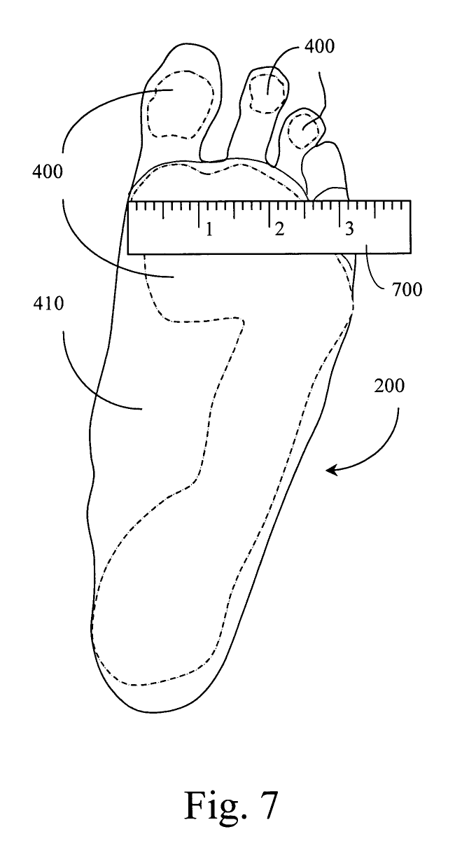

1. The length, L 500, of the foot from the heel to the longest toe is measured. Such a measurement, and following measurements, may be by ruler (see FIG. 7), computer aided scale, or other instruments of accurate measurement.

2. The length, LB 505, of the foot from the heel thereof to the ball of the foot is measured.

3. The width of the foot from the first metatarsal joint 510 to the fifth metatarsal joint 515 is measured.

4. The position of the metatarsal heads 520 is observed by visual means.

5. The position of the inner longitudinal arch (navicular) 525 is observed.

6. The position of the outer longitudinal arch (cuboid) 530 is observed.

7. Whether or not any of the metatarsal heads indicated generally at 520, are “dropped” is noted. This can be determined by visual observation of coloration of feet and stress points at met heads.

8. Whether or not planter calluses exist on the bottom of the foot can be ascertained by visual observations.

9. Plantar abnormalities and their positions, if any, are ascertained by visual observations such as neuromas, bunions, warts, missing toes, calluses, ingrown toenails, hammertoes, corns and others. Can see muscle tone which aids in showing weakness in arches. Weight distribution is shown by coloration and pressure points, which aids in determining degree of correction of arches.

10. The presence of hallux valgus (bunion) is noted by visual observations and the degree of forefoot eversion.

11. Whether or not a “Taylor's” bunion is present is noted by observation. This bunion is found off the little toe area 535.

12. Visual type observations are made of the inside of the arch 525 to indicate the presence and degree of pronation and the degree of forefoot eversion.

13. The presence and degree of suppination, always found at the outer side of the arch as indicated generally at 540, is observed along with degree of forefoot inversion.

14. The position of the toes is visually observed.

15. The entire images are analyzed to note any other type of plantar defects.

16. The position of the calcaneous is observed to check for pronation and supination around the posterior section 545.

17. Visual inspection for Morton's toe to Morton's neuroma. Morton's toe being seen as a shortened great toe 550 and a longer second toe 555. Morton's neuroma is seen between third 560 and fourth 565 toes as a color variation and indentation.

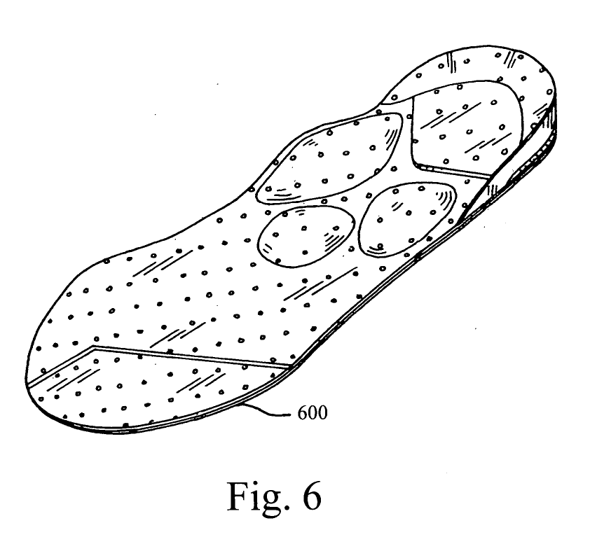

After careful analysis of the resulting measurements, visual observations, and color comparisons, the resulting data plus corrective measures are utilized in the laying out of the dimensions and sizes of materials for building corrective footwear such as a corrective arch support, orthotic, corrective shoe or sandal for the right foot 200. An example of an end result is shown as an orthotic 600 in FIG. 6. It should not be overlooked, however, that the measurements, such as shown in FIG. 7, and observations made and taken from the electronic image of the right foot 200 were corrected prior to the formation of the arch support so as to provide an arch support to restore normal foot balance to the right foot 200.

Incorporation-by-reference is hereby made to the computer CD submitted with this application. A directory listing for the CD is included in the appendix. The files included on the CD are briefly described in the appendix.

The above embodiment is the preferred embodiment, but this invention is not limited thereto. It is, therefore, obvious that many modifications and variations of the present invention are possible in light of the above teachings. It is, therefore, to be understood that within the scope of the appended claims, the invention may be practiced otherwise than as specifically described.

### Appendix

1. Computer Compatibility: IBM-PC
2. Operating System Compatibility: Windows 98, Windows ME, Windows 2000, Windows XP
3. Compression: Uncompressed data

The actual creation date of the attached CD-R is Jun. 20, 2003

The following files contain program code for all the screens. It controls the flow and execution of the program.

Associate directory

| | | | |
|---|---|---|---|
| Appcode.bas | 14 KB | 3/10/2003 | 12.11.36 PM |
| center.bas | 1 KB | 2/25/2003 | 11.32.08 AM |
| CFASScan.cls | 8 KB | 2/26/2003 | 02.35.00 PM |
| CheckFile.bas | 3 KB | 2/25/2003 | 11.32.08 AM |
| CurrencyPainter.cls | 2 KB | 2/25/2003 | 11.32.14 AM |
| frmAbout.frm | 13 KB | 2/25/2003 | 01.06.20 PM |
| frmAddCharges.frm | 6 KB | 2/25/2003 | 11.32.16 AM |
| frmBrowser.frm | 6 KB | 3/10/2003 | 12.14.26 PM |
| frmCashMarkup.frm | 2 KB | 2/25/2003 | 11.32.18 AM |
| frmDocPaymentScreen.frm | 84 KB | 2/26/2003 | 03.12.46 PM |
| frmDocSearch.frm | 77 KB | 2/25/2003 | 01.06.20 PM |
| frmDoctorCCInfo.frm | 28 KB | 2/26/2003 | 03.12.46 PM |
| frmDoctorChange.frm | 26 KB | 2/26/2003 | 03.12.46 PM |
| frmDrSetup.frm | 60 KB | 2/26/2003 | 03.12.46 PM |
| frmFeet.frm | 3 KB | 2/25/2003 | 11.32.20 AM |
| frmHelpMeImBlind.frm | 2 KB | 2/25/2003 | 11.32.20 AM |
| frmLicenseWindow.frm | 3 KB | 3/14/2003 | 02.49.12 PM |
| frmMsgBox.frm | 8 KB | 2/26/2003 | 02.35.00 PM |
| frmOrderDetails.frm | 84 KB | 2/26/2003 | 02.34.46 PM |
| frmOrderInfo1.frm | 71 KB | 2/26/2003 | 03.12.44 PM |
| frmOrderInfo2.frm | 31 KB | 2/26/2003 | 02.34.58 PM |
| frmOrderInfo3.frm | 31 KB | 2/26/2003 | 03.12.48 PM |
| frmOrderInfo4.frm | 59 KB | 3/10/2003 | 12.11.44 PM |
| frmOrderInfo5.frm | 62 KB | 2/26/2003 | 03.12.44 PM |
| frmOrderInfo6.frm | 64 KB | 2/26/2003 | 02.34.52 PM |
| frmOrderInfo7.frm | 82 KB | 3/10/2003 | 12.11.44 PM |
| frmOrderInfo8.frm | 60 KB | 3/10/2003 | 12.11.44 PM |
| frmOrderPrint.frm | 9 KB | 3/10/2003 | 12.11.44 PM |
| frmOrderRecieved.frm | 3 KB | 2/25/2003 | 01.06.22 PM |
| FrmPatienReport.frm | 8 KB | 2/26/2003 | 02.34.44 PM |
| frmPricing.frm | 22 KB | 2/26/2003 | 02.35.00 PM |
| frmProblems.frm | 13 KB | 2/26/2003 | 03.12.50 PM |
| frmPurge.frm | 10 KB | 2/26/2003 | 02.35.02 PM |
| frmRasChoice.frm | 4 KB | 2/26/2003 | 02.35.00 PM |
| frmResend.frm | 4 KB | 2/26/2003 | 02.35.00 PM |
| frmReviewOrderHist.frm | 15 KB | 2/26/2003 | 02.34.54 PM |
| frmReviewSavedOrders.frm | 23 KB | 2/26/2003 | 03.12.48 PM |
| frmSearch.frm | 54 KB | 2/26/2003 | 02.34.54 PM |
| frmShoppingCart.frm | 15 KB | 2/26/2003 | 02.34.30 PM |
| frmSummaryScreen.frm | 114 KB | 3/12/2003 | 11.19.44 AM |
| frmUnsentOrders.frm | 17 KB | 2/26/2003 | 02.34.54 PM |
| frmViewDoctorCost.frm | 27 KB | 2/26/2003 | 03.12.48 PM |
| mdiMain.frm | 26 KB | 3/12/2003 | 03.30.22 PM |
| modDBConnections.bas | 1 KB | 2/26/2003 | 02.35.00 PM |
| modKernel32.bas | 2 KB | 3/10/2003 | 12.11.44 PM |
| modTracing.bas | 3 KB | 2/25/2003 | 11.32.32 AM |
| modUser32.bas | 2 KB | 2/25/2003 | 11.32.32 AM |
| prjAssociate.vbp | 5 KB | 3/14/2003 | 02.18.48 PM |
| prjAssociate.vbw | 2 KB | 3/14/2003 | 02.38.14 PM |
| RegistryUtils.bas | 9 KB | 2/25/2003 | 11.32.40 AM |

The following files are binary files containing various static images used in displaying forms.

Associate\

| | | | |
|---|---|---|---|
| Associate.atb | 147 KB | 2/25/2003 | 11.32.16 AM |
| frmAbout.frx | 77 KB | 2/25/2003 | 01.06.20 PM |
| frmAddCharges.frx | 74 KB | 2/25/2003 | 11.32.16 AM |
| frmBrowser.frx | 144 KB | 3/10/2003 | 12.14.26 PM |
| frmDocPaymentScreen.frx | 201 KB | 2/26/2003 | 03.12.46 PM |
| frmDocSearch.frx | 74 KB | 2/25/2003 | 01.06.20 PM |
| frmDoctorCCInfo.frx | 73 KB | 2/26/2003 | 03.12.46 PM |
| frmDoctorChange.frx | 79 KB | 2/26/2003 | 03.12.46 PM |
| frmDrSetup.frx | 125 KB | 2/26/2003 | 03.12.46 PM |
| frmLicenseWindow.frx | 7 KB | 3/14/2003 | 02.49.12 PM |
| frmMsgBox.frx | 123 KB | 2/26/2003 | 02.35.00 PM |
| frmOrderDetails.frx | 101 KB | 2/26/2003 | 02.34.46 PM |
| frmOrderInfo1.frx | 178 KB | 2/26/2003 | 03.12.44 PM |
| frmOrderInfo2.frx | 751 KB | 2/26/2003 | 02.34.58 PM |
| frmOrderInfo3.frx | 123 KB | 2/26/2003 | 03.12.48 PM |
| frmOrderInfo4.frx | 223 KB | 2/26/2003 | 03.12.44 PM |
| frmOrderInfo5.frx | 169 KB | 2/26/2003 | 03.12.44 PM |
| frmOrderInfo6.frx | 123 KB | 2/26/2003 | 02.34.52 PM |
| frmOrderInfo7.frx | 222 KB | 2/26/2003 | 03.12.48 PM |
| frmOrderInfo8.frx | 125 KB | 2/26/2003 | 02.34.22 PM |
| frmOrderPrint.frx | 123 KB | 2/25/2003 | 01.06.22 PM |
| frmOrderRecieved.frx | 73 KB | 2/25/2003 | 01.06.22 PM |
| frmPricing.frx | 49 KB | 2/26/2003 | 02.35.00 PM |
| frmProblems.frx | 49 KB | 2/26/2003 | 03.12.50 PM |
| frmPurge.frx | 73 KB | 2/26/2003 | 02.35.02 PM |
| frmRasChoice.frx | 49 KB | 2/26/2003 | 02.35.00 PM |
| frmReviewOrderHist.frx | 100 KB | 2/26/2003 | 02.34.54 PM |
| frmReviewSavedOrders.frx | 175 KB | 2/26/2003 | 03.12.48 PM |
| frmSearch.frx | 98 KB | 2/26/2003 | 02.34.54 PM |
| frmShoppingCart.frx | 73 KB | 2/26/2003 | 02.34.30 PM |
| frmSummaryScreen.frx | 249 KB | 2/26/2003 | 03.12.42 PM |
| frmUnsentOrders.frx | 101 KB | 2/26/2003 | 02.34.54 PM |
| frmViewDoctorCost.frx | 49 KB | 2/26/2003 | 03.12.48 PM |
| mdiMain.frx | 1413 KB | 3/12/2003 | 03.30.22 PM |

The following file is used for long term data storage. All order and patient information is stored in this file.

Associate\

| | | | |
|---|---|---|---|
| Associate.mdb | 940 KB | 3/12/2003 | 03.42.18 PM |

The following file is contains registry settings used by program.

Associate\

| | | | |
|---|---|---|---|
| Associate.reg | 1 KB | 3/10/2003 | 12.11.38 PM |

The following files contain the code and binaries used to display and print various reports.

Associate\

| | | | |
|---|---|---|---|
| CrystalReport1.Dsr | 1 KB | 3/10/2003 | 12.11.38 PM |
| CrystalReport1.dsx | 15079 KB | 3/10/2003 | 12.11.42 PM |
| CrystalReport2.Dsr | 1 KB | 3/10/2003 | 12.11.42 PM |
| CrystalReport2.dsx | 131 KB | 3/10/2003 | 12.11.42 PM |
| CrystalReport3.DCA | 277 KB | 3/10/2003 | 12.11.42 PM |
| CrystalReport3.Dsr | 1 KB | 3/10/2003 | 12.11.42 PM |
| CrystalReport3.dsx | 131 KB | 3/10/2003 | 12.11.42 PM |
| CrystalReport4.Dsr | 1 KB | 3/10/2003 | 12.11.42 PM |
| CrystalReport4.dsx | 58 KB | 3/10/2003 | 12.11.42 PM |
| CrystalReport5.Dsr | 1 KB | 3/10/2003 | 12.11.44 PM |
| CrystalReport5.dsx | 33 KB | 3/10/2003 | 12.11.44 PM |
| DataEnvironment1.DCA | 30 KB | 2/25/2003 | 11.32.14 AM |
| DoctorEvalHistory.rpt | 33 KB | 2/25/2003 | 11.32.14 AM |
| DoctorOrderHistory.rpt | 57 KB | 2/25/2003 | 11.32.14 AM |
| Invoice.rpt | 130 KB | 2/25/2003 | 11.32.30 AM |
| InvoiceShopCart.rpt | 127 KB | 2/25/2003 | 11.32.30 AM |
| PatientInvoice.DCA | 226 KB | 2/25/2003 | 11.32.32 AM |
| PatientReport.DCA | 20837 KB | 2/25/2003 | 11.32.38 AM |
| PatientReport.rpt | 998 KB | 2/25/2003 | 11.32.40 AM |

Associate\GRID PICTURES

| | | | |
|---|---|---|---|
| Grand Slam Greta_{—} | 651 KB | 2/25/2003 | 11.32.44 AM |
| Norm.bmp | | | |
| Grand Slam Greta_{—} | 651 KB | 2/25/2003 | 11.32.44 AM |
| Pro.bmp | | | |
| Grand Slam Greta_{—} | 651 KB | 2/25/2003 | 11.32.46 AM |
| Sup.bmp | | | |
| GrandSlamGary_{—} | 651 KB | 2/25/2003 | 11.32.46 AM |
| Norm.bmp | | | |
| GrandSlamGary_{—} | 651 KB | 2/25/2003 | 11.32.46 AM |
| pron.bmp | | | |
| GrandSlamGary_{—} | 651 KB | 2/25/2003 | 11.32.46 AM |
| sup.bmp | | | |

Contains license information.

Associate\

| | | | |
|---|---|---|---|
| License.txt | 19 KB | 3/14/2003 | 02.48.16 PM |

Icon file—desktop shortcut image to program

Associate\

| | | | |
|---|---|---|---|
| Scanner1.ico | 7 KB | 3/10/2003 | 12.11.44 PM |

These are image files used when displaying images on the screen

Associate\Backgrounds

| | | | |
|---|---|---|---|
| BroFeet.jpg | 164 KB | 2/25/2003 | 11.32.40 AM |
| DSCF2579.jpg | 551 KB | 2/25/2003 | 11.32.40 AM |
| Normal-Foot.jpg | 211 KB | 2/25/2003 | 11.32.40 AM |
| ORTH_04.jpg | 356 KB | 2/25/2003 | 11.32.42 AM |
| Pronation.jpg | 208 KB | 2/25/2003 | 11.32.42 AM |
| SneakerCoupleDog_{—} | 413 KB | 2/25/2003 | 11.32.42 AM |
| SHOT4E.jpg | | | |
| Supination.jpg | 208 KB | 2/25/2003 | 11.32.42 AM |

These files are images used on the product order page. Some are images of product. Some are colors. Some are features included in various products.

Associate\GRID PICTURES

| | | | |
|---|---|---|---|
| 2003Logo_FLI_RGB_300dpi.jpg | 71 KB | 12/17/2002 | 02.12.18 PM |
| Associate Screen.bmp | 1406 KB | 1/13/2003 | 09.51.54 AM |
| BajaStrap.jpg | 3 KB | 2/25/2003 | 11.32.42 AM |
| black.jpg | 1 KB | 2/25/2003 | 11.32.42 AM |
| Bolt.jpg | 1 KB | 2/25/2003 | 11.32.42 AM |
| brown.jpg | 1 KB | 2/25/2003 | 11.32.42 AM |
| charcole.jpg | 1 KB | 2/25/2003 | 11.32.42 AM |
| chestnut.jpg | 1 KB | 2/25/2003 | 11.32.42 AM |
| CheyenneStrap.jpg | 2 KB | 2/25/2003 | 11.32.42 AM |
| Chocolate.jpg | 1 KB | 2/25/2003 | 11.32.44 AM |
| Comfort.jpg | 1 KB | 2/25/2003 | 11.32.44 AM |
| Drop.jpg | 3 KB | 2/25/2003 | 11.32.44 AM |
| Energy.jpg | 1 KB | 2/25/2003 | 11.32.44 AM |
| Feather.jpg | 3 KB | 2/25/2003 | 11.32.44 AM |
| GCS.jpg | 1 KB | 2/25/2003 | 11.32.44 AM |
| GCSX.jpg | 1 KB | 2/25/2003 | 11.32.44 AM |
| GraniteStrap.jpg | 2 KB | 2/25/2003 | 11.32.46 AM |
| InactiveGridPicture.jpg | 2 KB | 2/25/2003 | 11.32.46 AM |
| IntelliTemp.jpg | 1 KB | 2/25/2003 | 11.32.46 AM |
| Laces.jpg | 1 KB | 2/25/2003 | 11.32.46 AM |
| Legend.jpg | 32 KB | 2/25/2003 | 11.32.48 AM |
| LightBulb.jpg | 3 KB | 2/25/2003 | 11.32.48 AM |
| LightningBolt.jpg | 3 KB | 2/25/2003 | 11.32.48 AM |
| MenNoLace.jpg | 1 KB | 2/25/2003 | 11.32.48 AM |
| MenShoeIcon.jpg | 3 KB | 2/25/2003 | 11.32.48 AM |
| Moist.jpg | 1 KB | 2/25/2003 | 11.32.48 AM |
| MoistureResistant.jpg | 1 KB | 2/25/2003 | 11.32.48 AM |
| onyx.jpg | 1 KB | 2/25/2003 | 11.32.48 AM |
| OpenHeel.jpg | 1 KB | 2/25/2003 | 11.32.48 AM |
| OpenHeelIcon.jpg | 3 KB | 2/25/2003 | 11.32.48 AM |
| Phase3 Screen.bmp | 3164 KB | 2/25/2003 | 11.32.50 AM |
| PosturePicture.bmp | 651 KB | 2/25/2003 | 11.32.50 AM |
| Propacel.jpg | 1 KB | 2/25/2003 | 11.32.50 AM |
| sand.jpg | 1 KB | 2/25/2003 | 11.32.50 AM |
| SantaFeStrap.jpg | 2 KB | 2/25/2003 | 11.32.50 AM |
| ScanFeet Button.jpg | 9 KB | 1/24/2003 | 10.57.02 AM |
| Scanner.ico | 4 KB | 2/9/2003 | 09.52.08 PM |
| Scanner.jpg | 1 KB | 1/24/2003 | 12.10.50 PM |
| ShoppingCart.bmp | 1 KB | 2/25/2003 | 11.32.50 AM |
| Smart.jpg | 1 KB | 2/25/2003 | 11.32.50 AM |
| SneakerIcon.jpg | 3 KB | 2/25/2003 | 11.32.50 AM |
| StanceGuard.jpg | 1 KB | 2/25/2003 | 11.32.50 AM |
| sunset_blue.jpg | 1 KB | 2/25/2003 | 11.32.52 AM |
| tan.jpg | 1 KB | 2/25/2003 | 11.32.52 AM |
| Tough.jpg | 1 KB | 2/25/2003 | 11.32.52 AM |
| ToughIcon.jpg | 3 KB | 2/25/2003 | 11.32.52 AM |
| Umbrella.jpg | 3 KB | 2/25/2003 | 11.32.52 AM |
| white.jpg | 1 KB | 2/25/2003 | 11.32.52 AM |
| WomenNoLace.jpg | 1 KB | 2/25/2003 | 11.32.52 AM |
| WomenShoeIcon.jpg | 3 KB | 2/25/2003 | 11.32.52 AM |
| Zorbacel.jpg | 1 KB | 2/25/2003 | 11.32.52 AM |

Associate\GRID PICTURES\ORTHOTICS

| | | | |
|---|---|---|---|
| 101.jpg | 8 KB | 2/25/2003 | 11.32.52 AM |
| 105.jpg | 9 KB | 2/25/2003 | 11.32.52 AM |
| 112.jpg | 8 KB | 2/25/2003 | 11.32.54 AM |
| 136.jpg | 13 KB | 2/25/2003 | 11.32.54 AM |
| 1371.gif | 15 KB | 2/25/2003 | 11.32.54 AM |
| 1373.gif | 34 KB | 2/26/2003 | 12.08.38 PM |
| 1374.gif | 33 KB | 2/26/2003 | 12.08.44 PM |
| 138.jpg | 8 KB | 2/25/2003 | 11.32.54 AM |
| 1407.jpg | 9 KB | 2/25/2003 | 11.32.54 AM |
| 1408.jpg | 9 KB | 2/25/2003 | 11.32.54 AM |
| 1409.jpg | 15 KB | 2/25/2003 | 11.32.54 AM |
| 1410.jpg | 15 KB | 2/25/2003 | 11.32.54 AM |
| 142.jpg | 27 KB | 2/25/2003 | 11.32.54 AM |
| 1426.jpg | 13 KB | 2/25/2003 | 11.32.54 AM |
| 1427.jpg | 18 KB | 2/25/2003 | 11.32.54 AM |
| 1435.jpg | 20 KB | 2/25/2003 | 11.32.54 AM |
| 1436.jpg | 12 KB | 2/25/2003 | 11.32.56 AM |
| 144.jpg | 27 KB | 2/25/2003 | 11.32.56 AM |
| 1440.jpg | 10 KB | 2/25/2003 | 11.32.56 AM |
| 1441.jpg | 13 KB | 2/25/2003 | 11.32.56 AM |
| 1442.jpg | 17 KB | 2/25/2003 | 11.32.56 AM |
| 1443.jpg | 16 KB | 2/25/2003 | 11.32.56 AM |
| 1444.jpg | 15 KB | 2/25/2003 | 11.32.56 AM |
| 1445.jpg | 16 KB | 2/25/2003 | 11.32.56 AM |
| 1446.jpg | 16 KB | 2/25/2003 | 11.32.56 AM |
| 1447.jpg | 16 KB | 2/25/2003 | 11.32.56 AM |
| 1453.jpg | 18 KB | 2/25/2003 | 11.32.56 AM |
| 1454.jpg | 20 KB | 2/25/2003 | 11.32.58 AM |
| 1455.jpg | 20 KB | 2/25/2003 | 11.32.58 AM |
| 1456.jpg | 11 KB | 2/25/2003 | 11.32.58 AM |
| 1457.jpg | 14 KB | 2/25/2003 | 11.32.58 AM |
| 1458.jpg | 622 KB | 2/25/2003 | 11.32.58 AM |
| 1460.jpg | 20 KB | 2/25/2003 | 11.32.58 AM |
| 1474.jpg | 14 KB | 2/25/2003 | 11.32.58 AM |
| 1475.jpg | 13 KB | 2/25/2003 | 11.32.58 AM |
| 1476.jpg | 16 KB | 2/25/2003 | 11.32.58 AM |
| 1477.jpg | 16 KB | 2/25/2003 | 11.32.58 AM |
| 1478.jpg | 13 KB | 2/25/2003 | 11.32.58 AM |
| 1479.jpg | 11 KB | 2/25/2003 | 11.32.58 AM |
| 1480.jpg | 15 KB | 2/25/2003 | 11.33.00 AM |
| 1481.jpg | 11 KB | 2/25/2003 | 11.33.00 AM |
| 153.jpg | 12 KB | 2/25/2003 | 11.33.00 AM |
| 1605_Olympian_White.gif | 18 KB | 2/25/2003 | 11.33.00 AM |
| 1607_Olympian_Black.gif | 24 KB | 2/25/2003 | 11.33.00 AM |
| 1640_PathFinder.gif | 23 KB | 2/25/2003 | 11.33.00 AM |
| 169.jpg | 11 KB | 2/25/2003 | 11.33.00 AM |
| 1710_Milano_Black.gif | 21 KB | 2/25/2003 | 11.33.00 AM |
| 177.jpg | 7 KB | 2/25/2003 | 11.33.00 AM |
| 179.jpg | 8 KB | 2/25/2003 | 11.33.00 AM |
| 1801_Montana_Brown.gif | 26 KB | 2/25/2003 | 11.33.00 AM |
| 1803_Montana_Black.gif | 24 KB | 2/25/2003 | 11.33.00 AM |
| 183.jpg | 11 KB | 2/25/2003 | 11.33.02 AM |
| 184.jpg | 16 KB | 2/25/2003 | 11.33.02 AM |
| 185.jpg | 15 KB | 2/25/2003 | 11.33.02 AM |
| 5thAve_32.jpg | 5 KB | 2/25/2003 | 11.33.02 AM |
| 5thAve_717x130.jpg | 25 KB | 2/25/2003 | 11.33.02 AM |
| 5thAve_916x150.jpg | 49 KB | 2/25/2003 | 11.33.02 AM |
| 5thAvenue.jpg | 5 KB | 2/25/2003 | 11.33.02 AM |
| 5thAvenueSubGroup.jpg | 1 KB | 2/25/2003 | 11.33.02 AM |
| Aquaguards.jpg | 8 KB | 2/25/2003 | 11.33.02 AM |
| AquaguardsSubGroup.jpg | 1 KB | 2/25/2003 | 11.33.02 AM |
| Basic_32.ico | 24 KB | 2/25/2003 | 11.33.02 AM |
| basic_32.jpg | 6 KB | 2/25/2003 | 11.33.02 AM |
| Basic_717x130.jpg | 36 KB | 2/25/2003 | 11.33.04 AM |
| Basic_916x150.jpg | 43 KB | 2/25/2003 | 11.33.04 AM |
| combo_717x425.jpg | 57 KB | 2/25/2003 | 11.33.04 AM |
| combo_916x575.jpg | 90 KB | 2/25/2003 | 11.33.04 AM |
| EnergyFlex.jpg | 9 KB | 2/25/2003 | 11.33.04 AM |
| EnergyFlexSubGroup.jpg | 1 KB | 2/25/2003 | 11.33.04 AM |
| EnergySubGroup.jpg | 1 KB | 2/25/2003 | 11.33.04 AM |
| Extreme_32.ico | 24 KB | 2/25/2003 | 11.33.04 AM |
| extreme_32.jpg | 6 KB | 2/25/2003 | 11.33.04 AM |
| Extreme_717x130.gif | 28 KB | 2/25/2003 | 11.33.04 AM |
| Extreme_717x130.jpg | 47 KB | 2/25/2003 | 11.33.04 AM |
| Extreme_717x130.png | 519 KB | 2/25/2003 | 11.33.06 AM |
| Extreme_916x150.jpg | 70 KB | 2/25/2003 | 11.33.06 AM |
| ExtremeEnergy.jpg | 9 KB | 2/25/2003 | 11.33.06 AM |
| ExtremeXC.jpg | 5 KB | 2/25/2003 | 11.33.06 AM |
| ExtremeXCSubGroup.jpg | 1 KB | 2/25/2003 | 11.33.06 AM |
| ExtremeXS.jpg | 8 KB | 2/25/2003 | 11.33.06 AM |
| ExtremeXSSubGroup.jpg | 1 KB | 2/25/2003 | 11.33.06 AM |
| ExtremeXT.jpg | 9 KB | 2/25/2003 | 11.33.06 AM |
| ExtremeXTSubGroup.jpg | 1 KB | 2/25/2003 | 11.33.06 AM |
| FirmFlex.jpg | 8 KB | 2/25/2003 | 11.33.06 AM |
| FirmFlexSubGroup.jpg | 1 KB | 2/25/2003 | 11.33.08 AM |
| Illusion.jpg | 6 KB | 2/25/2003 | 11.33.08 AM |
| IllusionSubGroup.jpg | 1 KB | 2/25/2003 | 11.33.08 AM |
| LadyLevelers.jpg | 6 KB | 2/25/2003 | 11.33.08 AM |
| LadyLevelersSubGroup.jpg | 1 KB | 2/25/2003 | 11.33.08 AM |
| MsLevelers.jpg | 7 KB | 2/25/2003 | 11.33.08 AM |
| MsLevelersSubGroup.jpg | 1 KB | 2/25/2003 | 11.33.08 AM |
| ParFlexPlus.jpg | 9 KB | 2/25/2003 | 11.33.08 AM |
| ParFlexPlusSubGroup.jpg | 1 KB | 2/25/2003 | 11.33.08 AM |
| Plus_32.ico | 24 KB | 2/25/2003 | 11.33.08 AM |
| plus_32.jpg | 5 KB | 2/25/2003 | 11.33.08 AM |
| Plus_717x130.jpg | 41 KB | 2/25/2003 | 11.33.08 AM |
| Plus_916x150.jpg | 48 KB | 2/25/2003 | 11.33.08 AM |
| PowerSolesPlus.jpg | 6 KB | 2/25/2003 | 11.33.10 AM |
| PowerSolesPlusSubGroup. | 1 KB | 2/25/2003 | 11.33.10 AM |
| jpg | | | |
| SirLevelers.jpg | 9 KB | 2/25/2003 | 11.33.10 AM |
| SirLevelersSubGroup.jpg | 1 KB | 2/25/2003 | 11.33.10 AM |
| SkiFlexPlus.jpg | 8 KB | 2/25/2003 | 11.33.10 AM |
| SkiFlexPlusSubGroup.jpg | 1 KB | 2/25/2003 | 11.33.10 AM |
| Softhotic.jpg | 8 KB | 2/25/2003 | 11.33.10 AM |
| SofthoticsSubGroup.jpg | 1 KB | 2/25/2003 | 11.33.10 AM |
| Special_717x130.jpg | 39 KB | 2/25/2003 | 11.33.10 AM |
| Special_717x130OLD.jpg | 38 KB | 2/25/2003 | 11.33.10 AM |
| Special_916x150.jpg | 49 KB | 2/25/2003 | 11.33.10 AM |
| Specialty_32.ico | 24 KB | 2/25/2003 | 11.33.10 AM |
| specialty_32.jpg | 6 KB | 2/25/2003 | 11.33.10 AM |
| SPS.jpg | 7 KB | 2/25/2003 | 11.33.10 AM |
| SPSSubGroup.jpg | 1 KB | 2/25/2003 | 11.33.10 AM |
| Ultra_32.ico | 24 KB | 2/25/2003 | 11.33.12 AM |
| ultra_32.jpg | 5 KB | 2/25/2003 | 11.33.12 AM |
| Ultra_717x130.jpg | 43 KB | 2/25/2003 | 11.33.12 AM |
| Ultra_916x150.jpg | 55 KB | 2/25/2003 | 11.33.12 AM |
| UltraCombo_717x425.jpg | 95 KB | 2/25/2003 | 11.33.12 AM |
| UltraCombo_916x575.jpg | 147 KB | 2/25/2003 | 11.33.12 AM |
| UltraDry.jpg | 9 KB | 2/25/2003 | 11.33.12 AM |
| UltraDrySubGroup.jpg | 1 KB | 2/25/2003 | 11.33.12 AM |
| UltraEnergy.jpg | 10 KB | 2/25/2003 | 11.33.12 AM |
| UltraEnergySubGroup.jpg | 1 KB | 2/25/2003 | 11.33.12 AM |
| UltraStep.jpg | 8 KB | 2/25/2003 | 11.33.14 AM |
| UltraStepSubGroup.jpg | 1 KB | 2/25/2003 | 11.33.14 AM |
| UltratrekSubGroup.jpg | 1 KB | 2/25/2003 | 11.33.14 AM |
| YoungSoles_32.jpg | 1 KB | 2/25/2003 | 11.33.14 AM |
| YoungSoles_32OLD.jpg | 7 KB | 2/25/2003 | 11.33.14 AM |
| YoungSoles_32OLD2.jpg | 1 KB | 2/25/2003 | 11.33.14 AM |
| YoungSoles_717x130.jpg | 30 KB | 2/25/2003 | 11.33.14 AM |
| YoungSoles_916x150.jpg | 41 KB | 2/25/2003 | 11.33.14 AM |
| YoungUltra.jpg | 7 KB | 2/25/2003 | 11.33.14 AM |
| YoungUltraDry.jpg | 5 KB | 2/25/2003 | 11.33.14 AM |
| YoungUltraDrySubGroup.jpg | 1 KB | 2/25/2003 | 11.33.14 AM |
| YoungUltraSubGroup.jpg | 1 KB | 2/25/2003 | 11.33.14 AM |
| YoungXS.jpg | 5 KB | 2/25/2003 | 11.33.14 AM |
| YoungXSSubGroup.jpg | 1 KB | 2/25/2003 | 11.33.14 AM |

Associate\GRID PICTURES\SANDALTHOTICS

| | | | |
|---|---|---|---|
| 120_Malibu_SunsetBlue.gif | 16 KB | 2/25/2003 | 11.33.16 AM |
| 122_Malibu_Onyx.gif | 17 KB | 2/25/2003 | 11.33.16 AM |
| 123_Malibu_Chestnut.gif | 16 KB | 2/25/2003 | 11.33.16 AM |
| 126_Malibu_chocolate.gif | 16 KB | 2/25/2003 | 11.33.16 AM |
| 127_Jamaica_SunsetBlue.gif | 15 KB | 2/25/2003 | 11.33.16 AM |
| 129_Jamaica_onyx.gif | 17 KB | 2/25/2003 | 11.33.16 AM |
| 130_Jamaica_Chestnut.gif | 16 KB | 2/25/2003 | 11.33.16 AM |
| 133_Jamaica_Chocolate.gif | 18 KB | 2/25/2003 | 11.33.16 AM |
| 1501_Capetown_SunsetBlue.gif | 18 KB | 2/25/2003 | 11.33.16 AM |
| 1503_Capetown_Onyx.gif | 18 KB | 2/25/2003 | 11.33.16 AM |
| 1504_Capetown_Chestnut.gif | 17 KB | 2/25/2003 | 11.33.16 AM |
| 1507_Capetown_Chocolate.gif | 20 KB | 2/25/2003 | 11.33.16 AM |
| 1508.bmp | 68 KB | 2/25/2003 | 11.33.18 AM |
| 1508_Capetown_Sand.gif | 17 KB | 2/25/2003 | 11.33.18 AM |
| 1520_UTrek_Granite.gif | 21 KB | 2/25/2003 | 11.33.18 AM |
| 1521_UTrek_Baja.gif | 24 KB | 2/25/2003 | 11.33.18 AM |
| 1522_UTrek_SantaFe.gif | 21 KB | 2/25/2003 | 11.33.18 AM |
| 1523_UTrek_Cheyenne.gif | 23 KB | 2/25/2003 | 11.33.18 AM |
| 1550_Hollander_Tan.gif | 16 KB | 2/25/2003 | 11.33.18 AM |
| 1551_Hollander_Brown.gif | 16 KB | 2/25/2003 | 11.33.18 AM |
| 1552_Hollander_Black.gif | 16 KB | 2/25/2003 | 11.33.18 AM |
| 160_Malibu_Sand.gif | 16 KB | 2/25/2003 | 11.33.18 AM |
| 163_Jamaica_Sand.gif | 15 KB | 2/25/2003 | 11.33.18 AM |
| 198_Explorer_Black.gif | 19 KB | 2/25/2003 | 11.33.18 AM |
| 199_Explorer_Brown.gif | 17 KB | 2/25/2003 | 11.33.20 AM |
| Baja_Strap.bmp | 33 KB | 2/25/2003 | 11.33.20 AM |
| Black.bmp | 6 KB | 2/25/2003 | 11.33.20 AM |
| Black.jpg | 2 KB | 2/25/2003 | 11.33.20 AM |
| Brown.bmp | 6 KB | 2/25/2003 | 11.33.20 AM |
| Brown.jpg | 2 KB | 2/25/2003 | 11.33.20 AM |
| Capetown.jpg | 6 KB | 2/25/2003 | 11.33.20 AM |
| CapetownSubGroup.jpg | 1 KB | 2/25/2003 | 11.33.20 AM |
| Chestnut_Suede.bmp | 6 KB | 2/25/2003 | 11.33.20 AM |
| Chestnut_Suede.jpg | 3 KB | 2/25/2003 | 11.33.20 AM |
| Cheyenne_Strap.bmp | 34 KB | 2/25/2003 | 11.33.20 AM |
| Chocolate.bmp | 6 KB | 2/25/2003 | 11.33.20 AM |
| Chocolate.jpg | 2 KB | 2/25/2003 | 11.33.20 AM |
| Explorer.jpg | 5 KB | 2/25/2003 | 11.33.22 AM |
| ExplorerBlack.jpg | 5 KB | 2/25/2003 | 11.33.22 AM |
| ExplorerBrown.jpg | 5 KB | 2/25/2003 | 11.33.22 AM |
| ExplorerSubGroup.jpg | 1 KB | 2/25/2003 | 11.33.22 AM |
| Granite_Strap.bmp | 40 KB | 2/25/2003 | 11.33.22 AM |
| Hollander.jpg | 5 KB | 2/25/2003 | 11.33.22 AM |
| HollanderSubGroup.jpg | 1 KB | 2/25/2003 | 11.33.22 AM |
| Jamaica.jpg | 6 KB | 2/25/2003 | 11.33.22 AM |
| JamaicaSubGroup.jpg | 1 KB | 2/25/2003 | 11.33.22 AM |
| Malibu.jpg | 6 KB | 2/25/2003 | 11.33.22 AM |
| MalibuColors.bmp | 21 KB | 2/25/2003 | 11.33.22 AM |
| MalibuSubGroup.jpg | 1 KB | 2/25/2003 | 11.33.22 AM |
| NoImage.jpg | 5 KB | 2/25/2003 | 11.33.22 AM |
| Onyx.bmp | 6 KB | 2/25/2003 | 11.33.24 AM |
| Onyx.jpg | 2 KB | 2/25/2003 | 11.33.24 AM |
| Sand_Suede.bmp | 6 KB | 2/25/2003 | 11.33.24 AM |
| Sand_Suede.jpg | 2 KB | 2/25/2003 | 11.33.24 AM |
| SandalColors.ai | 307 KB | 2/25/2003 | 11.33.24 AM |
| SandalColors.bmp | 125 KB | 2/25/2003 | 11.33.24 AM |
| SandalColors.eps | 688 KB | 2/25/2003 | 11.33.24 AM |
| SandalColors.jpg | 14 KB | 2/25/2003 | 11.33.24 AM |
| Sandals_32.ico | 24 KB | 2/25/2003 | 11.33.24 AM |
| sandals_32.jpg | 6 KB | 2/25/2003 | 11.33.24 AM |
| sandals_717x130.jpg | 43 KB | 2/25/2003 | 11.33.26 AM |
| Sandals_916x150.jpg | 64 KB | 2/25/2003 | 11.33.26 AM |
| SanteFe_Strap.bmp | 36 KB | 2/25/2003 | 11.33.26 AM |
| SunsetBlue.bmp | 6 KB | 2/25/2003 | 11.33.26 AM |
| SunsetBlue.jpg | 2 KB | 2/25/2003 | 11.33.26 AM |
| Tan.bmp | 6 KB | 2/25/2003 | 11.33.26 AM |
| Tan.jpg | 2 KB | 2/25/2003 | 11.33.26 AM |
| Ultratrek.jpg | 5 KB | 2/25/2003 | 11.33.26 AM |
| UltratrekSubGroup.jpg | 1 KB | 2/25/2003 | 11.33.26 AM |

Associate\GRID PICTURES\SHOETHOTICS

| | | | |
|---|---|---|---|
| 1605_Olympian_White.gif | 18 KB | 2/25/2003 | 11.33.26 AM |
| 1607_Olympian_Black.gif | 24 KB | 2/25/2003 | 11.33.26 AM |
| 1640_PathFinder.gif | 23 KB | 2/25/2003 | 11.33.26 AM |
| 1710_Milano_Black.gif | 21 KB | 2/25/2003 | 11.33.28 AM |
| 1801_Montana_Brown.gif | 26 KB | 2/25/2003 | 11.33.28 AM |
| 1803_Montana_Black.gif | 24 KB | 2/25/2003 | 11.33.28 AM |
| Milano.jpg | 4 KB | 2/25/2003 | 11.33.28 AM |
| MilanoSubGroup.jpg | 1 KB | 2/25/2003 | 11.33.28 AM |
| Montana.jpg | 5 KB | 2/25/2003 | 11.33.28 AM |
| MontanaSubGroup.jpg | 1 KB | 2/25/2003 | 11.33.28 AM |
| Olympian.jpg | 4 KB | 2/25/2003 | 11.33.28 AM |
| OlympianSubGroup.jpg | 1 KB | 2/25/2003 | 11.33.28 AM |
| Pathfinder.jpg | 5 KB | 2/25/2003 | 11.33.28 AM |
| PathfinderSubGroup.jpg | 1 KB | 2/25/2003 | 11.33.28 AM |
| ShoeColors.jpg | 2 KB | 2/25/2003 | 11.33.28 AM |
| Shoes_32.ico | 24 KB | 2/25/2003 | 11.33.28 AM |
| shoes_32.jpg | 5 KB | 2/25/2003 | 11.33.30 AM |
| shoes_717x130.jpg | 42 KB | 2/25/2003 | 11.33.30 AM |
| Shoes_916x150.jpg | 60 KB | 2/25/2003 | 11.33.30 AM |

These files are a C program used to keep the scanner from going into sleep mode during periods of inactivity.

Associate\KeepAlive

| | | | |
|---|---|---|---|
| enum.cpp | 55 KB | 3/13/2003 | 06.11.30 PM |
| enum.h | 5 KB | 3/13/2003 | 10.53.06 AM |
| Extras.h | 1 KB | 2/25/2003 | 12.27.28 PM |
| KeepAlive.aps | 40 KB | 2/28/2003 | 03.04.58 PM |
| KeepAlive.clw | 1 KB | 2/28/2003 | 03.04.58 PM |
| KeepAlive.cpp | 2 KB | 3/13/2003 | 05.51.28 PM |
| KeepAlive.dsp | 5 KB | 3/14/2003 | 09.21.32 AM |
| KeepAlive.dsw | 1 KB | 2/24/2003 | 03.48.00 PM |
| KeepAlive.h | 1 KB | 2/25/2003 | 12.26.20 PM |
| KeepAlive.ncb | 257 KB | 3/14/2003 | 09.21.32 AM |
| KeepAlive.opt | 53 KB | 3/14/2003 | 09.21.32 AM |
| KeepAlive.plg | 2 KB | 3/14/2003 | 09.19.42 AM |
| KeepAlive.positions | 1 KB | 3/14/2003 | 09.21.32 AM |
| KeepAlive.rc | 5 KB | 2/28/2003 | 03.04.58 PM |
| KeepAliveDlg.cpp | 8 KB | 3/14/2003 | 09.12.56 AM |
| KeepAliveDlg.h | 1 KB | 2/27/2003 | 10.40.02 AM |
| mssccprj.scc | 1 KB | 6/23/2003 | 12.35.08 PM |
| objtwain.cpp | 124 KB | 6/26/2002 | 11.54.46 AM |
| objtwain.h | 47 KB | 6/26/2002 | 11.54.46 AM |
| OBJTwainErrorCodes.h | 3 KB | 3/7/2003 | 12.58.10 PM |
| ReadMe.txt | 3 KB | 2/24/2003 | 11.52.22 AM |
| resource.h | 1 KB | 2/27/2003 | 11.55.08 AM |
| StdAfx.cpp | 1 KB | 2/24/2003 | 11.52.22 AM |
| StdAfx.h | 1 KB | 2/27/2003 | 12.03.54 PM |
| Subclass.cpp | 7 KB | 11/22/1998 | 12.16.16 PM |
| Subclass.h | 2 KB | 11/22/1998 | 12.16.16 PM |
| TrayIcon.cpp | 5 KB | 2/27/2003 | 01.48.16 PM |
| TrayIcon.h | 1 KB | 2/27/2003 | 11.29.16 AM |
| twain.h | 82 KB | 9/11/2000 | 08.48.18 AM |
| twcontrol.cpp | 85 KB | 2/25/2003 | 11.07.00 AM |
| twcontrol.h | 19 KB | 2/26/2003 | 11.57.54 AM |
| TWImageLayout.cpp | 1 KB | 2/25/2003 | 05.25.34 PM |
| TWImageLayout.h | 1 KB | 2/26/2003 | 12.52.48 PM |
| usbdesc.h | 9 KB | 7/26/2000 | 04.34.26 PM |
| usbview.h | 5 KB | 7/26/2000 | 04.34.32 PM |

Associate\KeepAlive\res

| | | | |
|---|---|---|---|
| KeepAlive.ico | 7 KB | 2/26/2003 | 03.36.36 PM |
| KeepAlive.rc2 | 1 KB | 2/24/2003 | 11.52.22 AM |
| KeepAlive1.ico | 7 KB | 2/27/2003 | 11.55.08 AM |

The compiled executable to keep scanner from going into sleep mode

Associate\KeepAlive\Release

| | | | |
|---|---|---|---|
| KeepAlive.exe | 40 KB | 03/14/2003 | 09.19.42 AM |

The user manual for software.

Associate\Manual

| | | | |
|---|---|---|---|
| The Associate User Guide.pdf | 330 KB | 06/25/2002 | 01.28.24 PM |

1. A method for obtaining measurements for constructing corrective orthotics using at least one flatbed scanner and one computer, the method comprising:
(a) taking at least one scan of a patient's foot, the patient standing normally with at least one foot on the at least one flatbed scanner; and
(b) using measurements, observation, and color differences from the scan and computer to obtain the measurements necessary for constructing corrective footwear.

2. The method of claim 1 including constructing corrective orthotics by forming one or more pieces of material into a corrective arch support, orthotic, shoe or sandal while employing said measurements to accommodate the arch support to the foot from which the scan was made.

3. The method of claim 1 wherein said measurements are made by using a mechanical device, electronic device, and/or calculations based on color variations.

4. An apparatus for obtaining measurements for constructing corrective orthotics and footwear comprising:
(a) at least one flatbed scanner;
(b) a computer operatively attached to the scanner; and
(c) software operatively attached to the computer for said computer for organizing scanned foot data for making measurements and observations of the scanned foot data for the purpose of constructing corrective orthotics and footwear.
